(19) United States
(12) Patent Application Publication
(10) Pub. No.: US 2003/0049228 A1
(43) Pub. Date: Mar. 13, 2003

(54) Expression of foreign genes from plant virus vectors

(76) Inventors: Simon Santa-Cruz, West Sussex (GB); Gregory P. Pogue, Vacaville, CA (US); Rachel L. Toth, Fife (GB); Sean Chapman, Fife (GB); Fiona Carr, Fife (GB)

Correspondence Address: HOWREY SIMON ARNOLD & WHITE, LLP, BOX 34, 301 RAVENSWOOD AVE., MENLO PARK, CA, 94025, US

(21) Appl. No.: 09/758,962

(22) Filed: Jan. 9, 2001

(51) Int. Cl. A61K 48/00; C07H 21/04; C12N 7/00; C07K 14/005; C12N 5/08; C12N 15/86

(52) U.S. Cl. 424/93.2; 435/235.1; 435/366; 536/23.72; 530/350; 435/456

(57) ABSTRACT

The invention provides an expression system for foreign peptides by using a polynucleotide which encodes a promoter which transcribes a mRNA containing at least two open reading frames, from which an internal ribosome entry site directs the translation of one of the open reading frames. In addition, the invention provides a method of expressing a foreign peptide in a host using the expression system of the invention.

### FIELD OF THE INVENTION

This present invention is related to the field of viral vectors that are capable of expressing an open reading frame in a host. In particular, this invention relates to the use of an internal ribosome entry site, which is inserted into a heterologous virus to obtain gene expression.

### BACKGROUND OF THE INVENTION

Plant virus-based vectors have a number of advantages as gene expression tools including the ability to direct rapid and high-level expression of foreign genes in mature, differentiated, plant tissue and have been used for a number of different applications. Reporter proteins expressed by viral genomes allow localization of virus infected cells [1-3] and can be used to study mutant phenotypes [4, 5]. Plant virus-based vectors are also used for the production of valuable foreign peptides and proteins in plants [6].

Plant virus-based vectors offer advantages over other more costly and less flexible protein production systems such as fermentation, and much research has focused on the development of DNA and RNA viruses as vectors for gene expression in plants. Most approaches for the expression of foreign genes from viral vectors rely on either expression of the foreign protein as a fusion to a viral protein [2, 7, 8], or from a duplicated subgenomic mRNA promoter [9, 10]. However, a disadvantage of the latter approach is that the duplicated sequence is prone to homologous recombination with the consequent loss of the inserted sequence [11]. Translation of most eukaryotic mRNAs occurs by the scanning mechanism in which the 40S ribosome subunit binds to a 5′ cap structure and then “scans” the mRNA until it reaches an AUG translation initiation codon in a favorable sequence context where translation begins [12].

An alternative to cap-dependent initiation of translation, involving direct recruitment of ribosomes to internal tracts within mRNAs, has been observed for some cellular and viral mRNAs. Specific sequences, termed “internal ribosome entry sites” (IRES), located upstream of AUG codons have been found to be involved in this process, however, the mechanisms of IRES action are not fully understood [13]. IRESs have been found in capped as well as uncapped viral RNA, they show no strong sequence homology and direct the translation of mRNAs with different functions, under different physiological conditions. Although some reports of IRES sequences in plant viruses have proven controversial [14], Ivanov et al. [15] demonstrated that the 148 nucleotide sequence (IREScp) upstream of the coat protein (CP) gene of a crucifer-infecting tobamovirus (crTMV) is capable of promoting internal initiation of translation of the CP in vitro, acting as an IRES. Skulachev et al. [16] subsequently showed that this 148 nucleotide sequence sequence, and sequences originating from the region upstream of the movement protein gene in both crTMV and tobacco mosaic virus strain U1, mediated expression of a 3′-proximal reporter gene in vivo, on transfection of tobacco protoplasts and particle bombardment of N. benthamiana leaves with bicistronic RNA transcripts. Potato Virus X (PVX) is a single stranded RNA virus [17] that has been used successfully as a vector for gene expression in plants using both protein fusion and duplicated promoter expression strategies [1, 18].

Here we disclose a novel strategy for the expression of proteins in plants using viral vectors containing an IRES sequence. The strategy employed exploits the observation that, for PVX, cell-to-cell movement is completely dependent on the presence of viral coat protein [1, 5, 19]. In order to test the ability of the crTMV IREScp to direct the expression of an internal open reading frame from a heterologous viral vector we have assessed a series of viral constructs that produce a bicistronic mRNA carrying the green fluorescent protein (GFP) open reading frame positioned upstream of the IREScp sequence and followed by the PVX CP coding sequence. Using this strategy the infectivity of the viral constructs could be determined by the expression of GFP, and cell-to-cell movement, resulting in multicellular infection foci expressing GFP, could determine expression of coat protein.

The constructs described here are useful to direct expression of a foreign nucleic acid sequence in a host in the absence of multiple subgenomic promoters in the virus expressing the foreign nucleic acid sequence. The foreign nucleic acid sequence could code for a pharmaceutical protein useful in protein replacement therapy, or to intervene in a metabolic pathway to improve the nutritional value of a crop or alter the oil content of the seed.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention provides for a polynucleotide comprising (1) an IRES nucleotide sequence, (2) an ORF encoding a peptide of interest, and (3) an ORF encoding a viral protein. An IRES nucleotide sequence, or an IRES sequence, is any nucleotide sequence that can direct the translation of an ORF within a mRNA, or promote internal translation of an ORF. This IRES nucleotide sequence can be the nucleotide sequence of any IRES found in nature, or part thereof that behaves as any IRES found in nature. The ORF encoding a peptide of interest and the ORF encoding a viral protein may be transcribed on a single mRNA or transcript or message or transcriptional product. The single mRNA or transcript or message or transcriptional product may be transcribed from a promoter. This promoter is functional in an appropriate cell and is operatively linked to 5′ of the ORF encoding a peptide of interest and the ORF encoding a viral protein. The IRES nucleotide sequence is located between the ORF encoding a peptide of interest and the ORF encoding a viral protein. The IRES nucleotide sequence is able to direct the translation of whichever ORF is 3′ to the IRES nucleotide sequence. In one embodiment the polynucleotide comprises, in an order from 5′ to 3′: a promoter, an ORF, an IRES nucleotide sequence, and another ORF. The ORFs may be the ORF of a peptide of interest or the ORF of a viral protein.

The peptide of interest can be any peptide that can be expressed in an appropriate host. The host can be any cell or whole organism. The cell may be part of a cell culture or tissue culture or tissue or organ or a whole organism. The cell may also be a modified cell, such a protoplast cell. The cell may be an animal or plant cell. The peptide may be non-native or foreign to the IRES nucleotide sequence, the viral protein, or vector construct, or the host organism. The peptide may a peptide with pharmceutically useful properties, such as for protein replacement therapy, or any useful biological activity. The peptide may be useful in intervening in a metabolic pathway of the host organism in order to improve the nutritional content of the host, to improve the growth of the host organism, to improve the disease or pest resistance of the host, or to alter any other desired characteristic of the host.

The IRES can be an IRES from any organism as long as that IRES is able to function as an IRES within a host. The IRES can be from an animal (such as a mammal), plant, or a virus. The IRES nucleotide sequence can be a viral IRES. In a preferred embodiment the IRES nucleotide sequence is an IRES of a crucifer-infecting tobamovirus.

The viral protein can be a viral coat protein. In a preferred embodiment the viral protein is a viral coat protein of a crucifer-infecting tobamovirus.

In one embodiment of the invention, there is a plurality of ORFs and IRES nucleotide sequences, each ORF having an IRES nucleotide sequence upstream of the ORF.

The subject polynucleotide may be part of a recombinant viral construct, a recombinant viral vector construct, a recombinant virus, or a host infected by a recombinant virus. The virus may be a plant virus. In a preferred embodiment the virus is a plant RNA virus. In a more preferred embodiment the virus is PVX.

### BRIEF DESCRIPTION OF THE FIGURES

FIG. 1 depicts a schematic representation of the genome organization of the PVX wild-type clone, TXS, and its derivatives. ORFs are indicated by boxes with the size of their predicted products or the names of the proteins they encode (GFP, green fluorescent protein. CP, coat protein). An arrow indicates the promoter that directs expression of the PVX CP and hairpins indicate stem loops. The internal ribosome entry site originating from upstream of the coat protein in the crucifer-infecting TMV strain is indicated by IRES. Positions of restriction enzyme sites used to produce the constructs are indicated.

FIG. 2A depicts confocal images of inoculated N. benthamiana leaf tissue, 5 days post infection, showing single cell infections with TXS.GFPΔCP.

FIG. 2B depicts confocal images of inoculated N. benthamiana leaf tissue, 5 days post infection, showing single cell infections with TXS.GFP-IREScpH-CP.

FIG. 2C depicts confocal images of inoculated N. benthamiana leaf tissue, 5 days post infection, showing multicellular infection foci with TXS.GFP.

FIG. 2D depicts confocal images of inoculated N. benthamiana leaf tissue, 5 days post infection, showing multicellular infection foci with TXS.GFP-IREScp-CP.

FIG. 2E depicts confocal images of inoculated N. benthamiana leaf tissue, 5 days post infection, showing multicellular infection foci with TXS.GFP-HIREScp-CP.

FIG. 2F depicts confocal images of inoculated N. benthamiana leaf tissue, 5 days post infection, showing multicellular infection foci with TXS.GFP-SERIcp-CP.

FIG. 3 depicts the nucleotide sequences, between the SalI and SacI restriction sites, which comprises the IRES sequence, of clones TX.GFP-IRESs-CP (SEQ ID NO: 1), TXS-HRES-CP (SEQ ID NO: 2), TXS.GFP-SERI-CP (SEQ ID NO: 3), and TXS.GFP-IRESmp-CP (SEQ ID NO: 4).

FIG. 4 depicts the alignment of the nucleotide sequences, between the SalI and SacI restriction sites, comprising the IREScp sequence, in the progenitor clone TXS.GFP-IRESs-CP (SEQ ID NO: 5) and its derivatives SC196 (SEQ ID NO: 6) and SC197 (SEQ ID NO: 7). Asterisks (“*”) indicate nucleotides that have been substituted. “D” indicates nucleotides that have been deleted.

### DESCRIPTION OF THE SPECIFIC EMBODIMENTS

Definitions and Abbreviations

Bicistronic means a cistron which contains at least two ORFs in a mRNA transcribed from a single promoter whereby at least two peptides are translated from the mRNA.

Plant virus-based vectors means an engineered plant virus which is capable of expressing a desired protein or trait in a host.

Expression means transcription, translation, protein synthesis, or any combination of transcription, translation, and protein synthesis.

Foreign gene means any gene that is not derived from or extracted from or native to a vector into which it is inserted.

Reporter protein means a protein which when expressed by viral genomes allow localization of virus-infected cells.

Host means a cell, tissue, organ, or organism capable expressing the ORFs of the subject polynucleotides. This term is intended to include prokaryotic and eukaryotic cells, organs, tissues or organisms, where appropriate. Bacteria, fungi, yeast, animal (cell, tissue, organ, or organism), and plant (cell, tissue, organ, or organism) are examples of a host.

Infection mean the ability of a virus to transfer its nucleic acid to a host or introduce a viral nucleic acid into a host, wherein the viral nucleic acid is replicated, viral proteins are synthesized, and new viral particles assembled. The term is also meant to include the ability of a selected nucleic acid sequence to integrate into a genome, chromosome or gene of a host or target organism.

Internal ribosome entry sites (IRES) means a nucleic acid sequence which involving direct recruitment of ribosomes to internal tracts within mRNAs. IRESs are an alternative to cap-dependent initiation of translation. Specific sequences, termed IRES direct the translation of mRNAs with different functions, under different physiological conditions.

IRESmp means nucleotide sequence (IRESmp) upstream of the movement protein (MP) gene of Tobamovirus, IRESmp is capable of promoting internal initiation of translation of the MP RNA in vitro, acting as an IRES.

IREScp, the 148 nucleotide sequence (IREScp) upstream of the coat protein (CP) gene of a crucifer-infecting tobamovirus (crTMV) is capable of promoting internal initiation of translation of the CP RNA in vitro, acting as an IRES. This sequence, and sequences originating from the region upstream of the movement protein gene in both crTMV and tobacco mosaic virus strain U1, mediated expression of a 3′-proximal reporter gene in vivo, on transfection of tobacco protoplasts and particle bombardment of N. benthamiana leaves with dicistronic RNA transcripts.

Heterologous viral vector means an engineered virus, which is not the origin of the IRES into which it is inserted. Similarly an IRES sequence which is heterologous to the viral vector is one which does not derive from the virus.

ORF or open reading frame means a nucleotide sequence encoding a series of sense codons that lack a terminating codon. The ORF may be encoded in any nucleic acid, including DNA or RNA, and the nucleic acid may be any form, including single-stranded or double-stranded. An ORF may encoded a peptide that is expressed and may be a gene.

The Invention

The present invention provides for a polynucleotide comprising (1) an IRES nucleotide sequence, (2) an ORF encoding a peptide of interest, and (3) an ORF encoding a viral protein. An IRES sequence, is defined as any nucleotide sequence that can direct the translation of an ORF within a mRNA, or promote internal translation of an ORF. One method by which an IRES nucleotide sequence may direct the translation of an ORF within a mRNA, or promote internal translation of an ORF, is by direct recruitment of ribosomes to internal tracts within mRNAs. An IRES sequence can be the nucleotide sequence of any IRES found in nature, or part thereof that behaves as any IRES found in nature. An IRES sequence can also be any nucleotide sequence that is synthetic or artificially designed that can direct the translation of an ORF within a mRNA. One of ordinary skill in the art can by performing the present experiments disclosed, with alterations of the IRES sequence, determine whether any nucleotide sequence can direct the translation of an ORF within a mRNA in an appropriate host. An IRES sequence can comprise the nucleotide sequence of SEQ ID NO: 1, SEQ ID NO: 2, SEQ ID NO: 3, SEQ ID NO: 4, SEQ ID NO: 5, or SEQ ID NO: 6, or any fragment thereof that can direct the translation of an ORF within a mRNA in an appropriate host.

The ORF encoding a peptide of interest and the ORF encoding a viral protein may be transcribed on a single mRNA or transcript or message or transcriptional product. The single mRNA or transcript or message or transcriptional product may be transcribed from a promoter. This promoter is functional in an appropriate cell and is operatively linked to 5′ of the ORF encoding a peptide of interest and the ORF encoding a viral protein. The IRES nucleotide sequence is located between the ORF encoding a peptide of interest and the ORF encoding a viral protein. The IRES nucleotide sequence is able to direct the translation of whichever ORF is 3′ to the IRES nucleotide sequence. In one embodiment the polynucleotide comprises, in an order from 5′ to 3′: a promoter, an ORF, an IRES nucleotide sequence, and another ORF. The ORFs may be the ORF of a peptide of interest or the ORF of a viral protein.

The peptide of interest can be any peptide that can be expressed in an appropriate host. The host can be any cell or whole organism. The cell may be part of a cell culture or tissue culture or tissue or organ or a whole organism. The cell may also be a modified cell, such a protoplast cell. The cell may be an animal or plant cell. The peptide may be non-native or foreign to the IRES nucleotide sequence, the viral protein, or vector construct, or the host organism. The peptide may a peptide with pharmceutically useful properties, such as for protein replacement therapy, or any useful biological activity. The peptide may be useful in intervening in a metabolic pathway of the host organism in order to improve the nutritional content of the host, to improve the growth of the host organism, to improve the disease or pest resistance of the host, or to alter any other desired characteristic of the host.

The IRES can be an IRES from any organism as long as that IRES is able to function as an IRES within a host. The IRES can be from an animal (such as a mammal), plant, or a virus. The IRES nucleotide sequence can be a viral IRES. In a preferred embodiment the IRES nucleotide sequence is an IRES of a plant virus. In a more preferred embodiment the IRES nucleotide sequence is an IRES of a plant RNA virus. In an even more preferred embodiment the IRES nucleotide sequence is an IRES of a tobamovirus. In an even further more preferred embodiment the IRES nucleotide sequence is an IRES of a crucifer-infecting tobamovirus.

The viral protein can be a viral coat protein. In a more preferred embodiment the viral protein is a viral coat protein of a plant virus. In an even more preferred embodiment the viral protein is a plant viral coat protein. In an even further more preferred embodiment the viral protein is coat protein of a crucifer-infecting tobamovirus.

The subject polynucleotide can comprise of the following components in the following order:

P-N-R-V or P-V-R-N

wherein “P” is the promoter, “N” is the ORF of the peptide of interest, “V” is the ORF of the viral protein, and “R” is the IRES nucleotide sequence. In addition the subject polynucleotide may have additional nucleotide sequences between each component and/or flanking the components, as long as these nucleotide sequences do not interfere with the transcription or translation or expression of the ORF of the peptide of interest and the ORF of the viral protein.

In another embodiment of the invention, there is a plurality of ORFs and IRES nucleotide sequences, in which the subject polynucleotide can comprise of the following components in the following order:

P-N-R-V-R₁-N₁-R₂-N₂- . . . -Rₙ-Nₙ or
P-V-R-N-R₁-N₁-R₂-N₂- . . . -Rₙ-Nₙ

wherein “P”, “N”, “R” and “V” are as defined above and “Rₙ” is any IRES nucleotide sequence (as exemplified by “R₁” and “R₂” above) and “Nₙ” is any ORF of any desired viral or non-viral peptide (as exemplified by “N₁” and “N₂” above), where “n” is an integer equal to one or greater. In this embodiment, there is a plurality of ORFs and IRES nucleotide sequences: each ORF having an IRES nucleotide sequence upstream of the ORF. In addition the subject polynucleotide may have additional nucleotide sequences between each component and/or flanking the components, as long as these nucleotide sequences do not interfere with the transcription or translation or expression of any ORF.

The subject polynucleotide can be part of a plasmid, vector, vector construct, viral vector construct, recombinant viral construct, or part of a viral genome. The subject polynucleotide or aforementioned plasmid, replicon, episome, vector, vector construct, viral vector construct, recombinant viral construct, or viral genome can be part of a virus, recombinant virus, viral particle, virion or the like. The virus, recombinant virus, viral particle, or virion can infect an appropriate host.

The virus may be a plant virus. In a preferred embodiment the virus is a plant RNA virus. In a more preferred embodiment the virus is a plant single-stranded RNA virus. In an even more preferred embodiment the virus is Potato Virus X (“PVX”). PVX is a single-stranded RNA virus that has been used successfully as a vector for gene expression in plants using both protein fusion and duplicated promoter expression strategies.

In one embodiment of the invention, an IRES is capable of directing the expression of an internal ORF in a heterologous viral vector.

In another embodiment of the invention, a viral vector construct is described which produces a bicistronic mRNA carrying an ORF positioned upstream of an IRES sequence and followed by a coat protein coding sequence. The ORF can be any gene to be expressed in a host, or it may be a reporter gene ORF by which the progress of coat protein expression and viral movement can be monitored.

In another embodiment, a viral vector construct, comprises: (1) the genome of a virus, and (2) an IRES sequence which is heterologous to the virus, the IRES sequence being inserted into the virus downstream of any desired gene or ORF and upstream of a virus coat protein gene, wherein the IRES sequence being inserted in the sense or antisense orientation. This viral construct might be one that produces a bicistronic mRNA.

In another embodiment, a viral vector construct, comprising: (1) the genome of a virus, (2) an IRES sequence which is heterologous to the virus, the IRES sequence being inserted into the virus downstream of any desired gene or ORF and upstream of a virus coat protein gene, wherein the IRES sequence being inserted in the sense or antisense orientation; and, (3) a stable stem loop structure inserted 5′ of an IRES sequence. Such a viral vector construct might give rise to one or more single cell infection sites or to systemic-host infection. A viral vector construct comprising a stem loop structure functions as a site for direct recruitment of ribosomes for initiation of translation. The stem loop structure can be immediately upstream of the IRES sequence. A viral vector construct having a stem loop immediately upstream of the IRES sequence leads to a reduction in coat protein expression levels. It may be useful to control of rate of system infection.

In another embodiment a viral vector construct comprises: (1) the genome of a virus; (2) an IRES sequence which is heterologous to the virus, the IRES sequence being inserted into the virus downstream of any desired gene or ORF and upstream of a virus coat protein gene, wherein the IRES sequence is inserted in the sense or antisense orientation; and, (3) a stable stem loop structure inserted 3′ of an IRES sequence. This construct is capable of preventing expression of the downstream CP ORF in the bicistronic mRNA. It is also able to effectively block scanning ribosomes.

The invention is exemplified by viral constructs comprising the nucleotide sequences of TXS, TXS.GPF, TXS.GPF-ΔCP, TXS.GPF-IRES-CP, TXS.GPF-IRESs-CP,TXS.GPF-HIRES-CP, TXS.GPF-IRESH-CP, TXS.GPF-SERI-CP, and TXS.GFP-IRESs(mp)-CP.

The provides for a virus, recombinant virus, viral particle, virion or the like comprising any of the aforementioned viral vector constructs or viral constructs.

The following examples further illustrate the present invention. These examples are intended merely to be illustrative of the present invention and are not to be construed as being limiting.

### EXAMPLES

Potato virus X (PVX)-based vector constructs were generated to investigate the use of an internal ribosome entry site (IRES) sequence to direct translation of a viral gene. An IRES sequence from a crucifer-infecting strain of tobacco mosaic virus was used to direct expression of the PVX CP. The IRES was inserted downstream of the gene encoding GFP and upstream of the PVX CP, in either sense or antisense orientation, such that CP expression depended on ribosome recruitment to the IRES. Stem loop structures were inserted at either the 3′-or 5′-end of the IRES sequence to investigate its mode of action. In vitro RNA transcripts were inoculated to Nicotiana benthamiana plants and protoplasts, levels of GFP and CP expression were analysed by ELISA and the rate of virus cell-to-cell movement was determined by confocal laser scanning microscope imaging of GFP expression. PVX CP was expressed, allowing cell-to-cell movement of virus, from constructs containing the IRES sequence in either sense or antisense orientation, and from the construct containing a stem loop structure at the 5′-end of the IRES sequence. No CP was expressed from a construct containing a stem loop at the 3′-end of the IRES sequence. Our results suggest that the IRES sequence is acting in vivo to direct expression of the 3′-proximal ORF in a bicistronic mRNA thereby demonstrating the potential of employing IRES sequences for the expression of foreign proteins from plant virus-based vectors.

Biological Deposits

The following plasmid vectors were deposited with ATCC, 10801 University Blvd., Manassas, Va. 20110, USA on Nov. 22, 2000: pHIRES-XCP (accession no. PTA-2717), pSERI-XCP (accession no. PTA-2718), pIRES-XCP (accession no. PTA-2719), pIRESx-XCP (accession no. PTA-2720), pTXS.GFP-IRESH-CP (accession no. PTA-2721), pTXS.GFP-SERI-CP (accession no. PTA-2722), pTXS.GFP-HIRES-CP (accession no. PTA-2723), pTXS.GFP-IRES-CP (accession no. PTA-2724), pTXS.GFP-IRESs-CP (accession no. PTA-2725), and pTXS.GFP-IRESs(mp)-CP (accession no. PTA-2726).

### EXAMPLE 1

Plasmid Constructions.

The plasmid pTXS, carrying a cDNA of the wild-type PVX genome, pTXS.GFP, in which GFP expression is under the transcriptional control of a duplicated subgenomic mRNA promoter, and pTXS.GFP-ΔCP, in which the CP sequence is deleted, have been described previously [5]. The plasmid pIRES-GUS carries the IREScp sequence positioned upstream of the P-glucuronidase (GUS) open reading frame [15].

Construction of pIRES-XCP.

PCR amplification was performed using pTXS as template with an upstream mutagenic primer designed to introduce an NcoI restriction site across the initiating AUG codon of the viral CP RNA and the universal reverse primer as the downstream oligonucleotide. Following digestion of the amplification product with NcoI and SacI, the resulting fragment, which spans the CP RNA sequence and 3′-untranslated region of PVX, was inserted into pIRES-GUS from which the GUS coding sequence had been removed by digestion with NcoI and SacI. The resulting plasmid, pIRES-XCP was the basis for all subsequent plasmid constructions.

Construction of pIRESs-XCP.

PCR amplification of pIRES-XCP was performed with a non-mutagenic upstream primer, that introduced an EcoRI site, and a mutagenic downstream primer that introduced a SacI site between the 3′ end of the IREScp sequence and the NcoI site. The amplification product was digested with EcoRI and NcoI prior to cloning of the fragment between the same sites, flanking the IREScp sequence, of pIRES-XCP to produce pIRESs-XCP.

Construction of pSERI-XCP with Anti-Sense IRES.

PCR amplification of pIRES-XCP was performed with mutagenic primers that introduced NcoI and EcoRI sites at the 5′ and 3′ ends of the IREScp sequence, respectively. The amplification product was digested with NcoI and EcoRI prior to cloning of the amplified fragment in reverse orientation between the same sites of pIRES-XCP to produce pSERI-XCP.

Construction of pHIRES-XCP with Stem Loop.

Sequence encoding a stem loop was introduced 5′ of the IREScp sequence by digestion of pIRES-XCP with EcoRI and ligation to a self-annealed oligonucleotide (5′-AAT-TCG-GAT-CCC-GGG-GGG-CCC-TAC-CGC-CGC-GGC-GGT-TAA-CCG-CCG-CGG-CGG-TAG-GGC-CCC-CCG-GGA-TCC-G-3′) (SEQ ID NO: 8) producing pHIRES-XCP.

Construction of Full-Length Clones.

Full-length clones were produced by digestion of the subclones pIRES-XCP, pIRESs-XCP, pSERI-XCP and pHIRES-XCP with SalI and SpeI, and cloning of the released fragments, encompassing the IREScp sequence, CP and 3′ untranslated region of PVX, between the same sites of pTXS.GFP to produce pTXS.GFP-IRES-CP, pTXS.GFP-IRESs-CP, pTXS.GFP-SERI-CP and pTXS.GFP-HIRES-CP respectively.

Construction of pTXS.GFP-IRESH-CP.

The plasmid pHIRES-XCP was digested with EcoRI to release the fragment encoding the stem loop and, after T4 DNA polymerase treatment to fill in the overhangs, this fragment was cloned into pTXS.GFP-IRESs-CP that had been digested with SacI and T4 DNA polymerase treated. The resulting plasmid, pTXS.GFP-IRESH-CP, thus contained the sequence encoding the stem loop 3′ of the IREScp sequence.

Construction of pIRESs(mp)-XCP.

A fragment encompassing nucleotides 4800 to 4874 was amplified from a full-length turnip vein clearing virus cDNA clone (Lartey, R. T., Voss, T. C., Melcher, U. Gene 1995. 166(2):331-2; Lartey, R. T., Lane, L. C., Melcher, U. Arch. Virol. 1994. 138(3-4):287-98) using mutagenic primers that introduced EcoRI, and SacI and NcoI sites at the 5′ and 3′ ends respectively. The amplification product was digested with EcoRI and NcoI prior to cloning between the same sites of pIRES-XCP to produce pIRESs(mp)-XCP.

Construction of Full Length Clone pTXS.GFP-IRESs(mp)-CP.

A full-length clone was produced (as with the other subclones) by digestion with SalI and SpeI, and cloning of the released fragment between the same sites of pTXS.GFP to produce pTXS.GFP-IRESs(mp)-CP.

In Vitro Transcription and Plant Inoculation.

All plasmids were linearized with SpeI prior to in vitro transcription using a T7 mMESSAGE mMACHINE™ kit (Ambion, Austin, Tex.) according to the manufacturer's instructions. Transcription reaction products were inoculated directly to aluminum oxide dusted leaves of N. benthamiana as described previously [5]. Two leaves were inoculated per plant, and each leaf was inoculated with the transcript products derived from 0.2 μg plasmid template.

Detection of Fluorescence and Measurement of Infection Foci.

Leaves were viewed under UV illumination (365 nm) generated from a Blak Ray B100-AP lamp (Ultraviolet Products, San Gabriel, Calif.). For confocal imaging 0.5 cm² squares of leaf tissue were cut, mounted in water and imaged using an MRC 1000 confocal laser-scanning microscope (Bio-Rad, Hercules, Calif.) as described previously [5]. The size of fluorescent infection foci was measured using COMOS software (Bio-Rad). An analysis of variance was carried out on area measurements of 20 separate infection foci for each construct, 5 days post inoculation (dpi). The least significant difference at a 5% level was calculated and used to identify groups of data showing statistically significant differences.

Protoplast Preparation and Transfection.

N. benthamiana plants (4-5 weeks old), grown for at least 10 days in a controlled environment room (16 h light, 22° C.), were used for preparation of mesophyll protoplasts according to the method of Power and Chapman [20]. Approximately 6×10⁵ protoplasts were electroporated with 10 μL of an in vitro transcription reaction as previously described [21]. Electroporated protoplast samples were incubated at approximately 20° C. with 16 h light (200 lux) and harvested after 48 h for protein quantification by ELISA.

ELISA.

Accumulation of GFP and CP in transfected protoplasts was determined by indirect triple antibody sandwich ELISA essentially as described by Clarke and Bar-Joseph [22]. ELISA plate wells (MaxiSorp, Nalgene Nunc International, Rochester, N.Y.) were coated with a monoclonal antibody raised against either GFP (antibody 3E6, Molecular Probes, Eugene, Oreg.) or PVX CP (antibody MAC58). Protoplasts from triplicate wells of a tissue culture plate were pooled, pelleted and the pellets ground in 400 μl PBS. Supernatants collected after brief centrifugation were used for ELISA. Bound CP and GFP were probed for with polyclonal antiserum raised against either PVX (Adgen, Auchincruive, U.K.) or GFP [18] and subsequently alkaline phosphatase conjugated antibody raised against rabbit IgG (Sigma, St Louis, Mo.). All antibodies were diluted 1:2000 for use. The ELISA reaction product (p-nitrophenyl) was quantified calorimetrically. Levels of CP and GFP were calculated from standards using Biolinx™ software (Dynatech Laboratories, Chantilly, Va.).

Results.

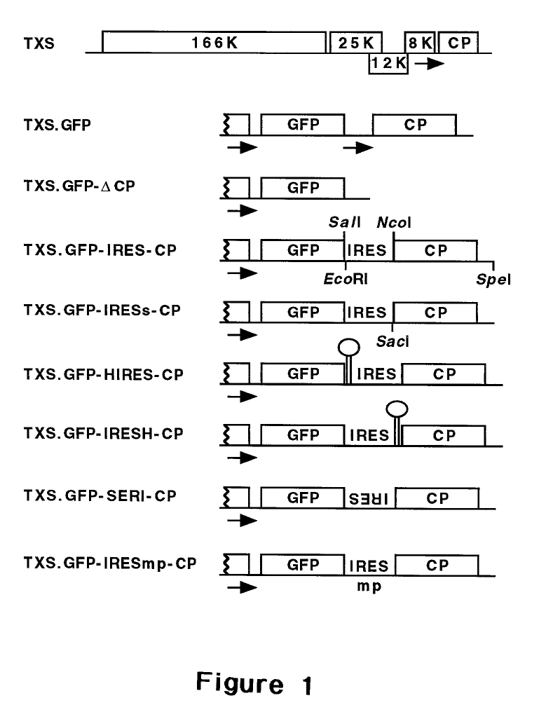

Five different IRES-containing constructs were produced (FIG. 1). In pTXS.GFP-IRES-CP, the IRES was introduced in the sense orientation between the GFP and CP coding sequences in order to allow the synthesis of a bicistronic subgenomic mRNA with coat protein expression dependent on recruitment of ribosomes to the IREScp sequence. A derivative of this clone, pTXS.GFP-IRESs-CP, with a unique SacI restriction enzyme recognition site between the IRES sequence and the PVX CP gene, was prepared to aid subsequent plasmid constructions.

In the clone pTXS.GFP-HIRES-CP, sequence encoding a stem loop structure (ΔG-90 kcal/mol), was inserted between the 3′-end of the GFP gene and the 5′-end of the IRES sequence, in order to block leaky scanning of ribosomes through the gfp gene. In pTXS.GFP-IRESH-CP the stem loop structure described above was positioned between the 3′ end of the IRES sequence and the CP coding sequence, in order to block scanning ribosomes and prevent translation of the CP. Thus, if the crTMV-derived sequence was acting as an IRES, the introduction of the stem loop at the 3′-end but not the 5′-end of the IRES would be expected to block CP expression. In the plasmid pTXS.GFP-SERI-CP, the IRES was placed in the opposite orientation in the expectation that IRES activity would be blocked and CP expression would not occur.

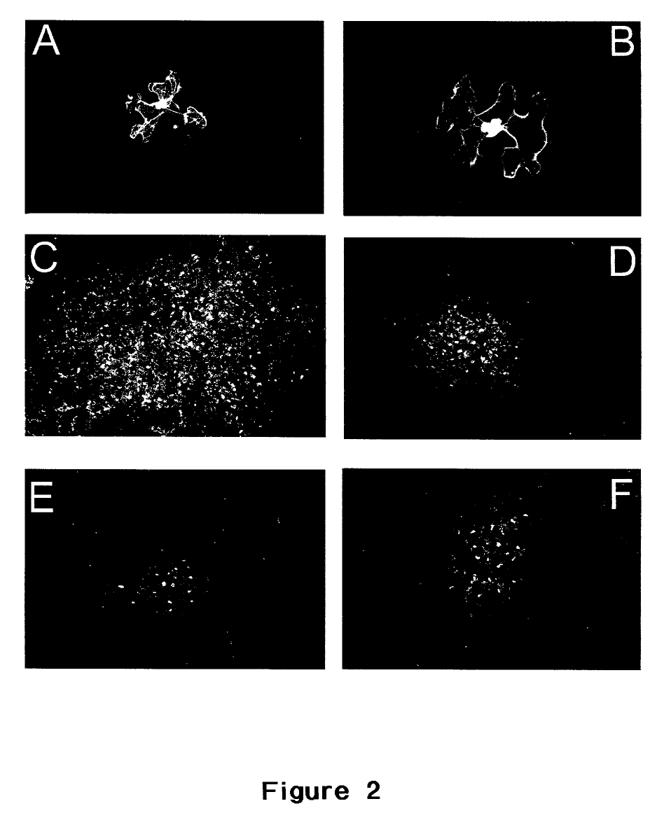

In vitro run-off transcripts, synthesized from the above plasmids and the control plasmids pTXS.GFP and pTXS.GFPΔCP, were infectious as determined by the expression of GFP, giving rise either to individual GFP expressing cells (TXS.GFP-IRESH-CP, pTXS.GFPΔCP) or multicellular infection sites (pTXS.GFP-IRES-CP, pTXS.GFP-IRESs-CP, pTXS.GFP-HIRES-CP, pTXS.GFP-SERI-CP and pTXS.GFP, FIG. 2).

Area measurements of multicellular, fluorescent, infection foci obtained with the different CP-expressing constructs, representative examples of which are shown in FIG. 2, indicated that the size of infection foci varied between the different constructs. The mean area of 20 infection foci, measured for each construct showing cell-to-cell movement at 5 dpi are shown in Table 1. Statistical analysis of the data showed that all the IRES-containing constructs displayed significantly slower cell-to-cell movement than the control TXS.GFP, in which the CP is translated directly from the 5′-end of a subgenomic mRNA. Furthermore, the infection foci produced by TXS.GFP-HIRES-CP were significantly smaller than those produced by TXS.GFP-IRES-CP, TXS.GFP-IRESs-CP and TXS.GFP-SERI-CP (Table 1).

TABLE 1
Measurement of fluorescent infection foci.*

| | mean area of fluorescent |
|---|---|
| inoculum | infection foci (mm²) |
| TXS.GFP | 4.90 |
| TXS.GFP-IRES-CP | 0.516 |
| TXS.GFP-IRESs-CP | 0.514 |
| TXS.GFP-SERI-CP | 0.461 |
| TXS.GFP-HIRES-CP | 0.257 |
| TXS.GFP-IRESH-CP | 0.0 |
| TXS.GFP-IRESs(mp)-CP | 0.271 |

*The areas of fluorescent lesions on inoculated leaves of N. benthamiana were measured 5 dpi. Mean areas in mm² are presented. Analysis of variance gave a least significant difference at the 5% level of 0.107.

The constructs were further analyzed in transcript-inoculated N. benthamiana protoplasts with TXS, TXS.GFP, and TXS.GFPΔCP as controls. The levels of GFP and CP accumulation in infected protoplasts were measured by ELISA at 2 dpi (Table 2). The results from this quantitative study of CP and GFP accumulation correlated well with the in planta observations. All of the constructs carrying the gfp gene, with the exception of TXS.GFPΔCP, gave rise to similar levels of GFP accumulation, while levels of CP expression were more variable. There was no detectable CP expression in infections with TXS.GFP-IRESH-CP, as expected from the in planta experiment where no cell to cell movement was observed. The other IRES-containing constructs accumulated lower levels of CP than the TXS and TXS.GFP controls. The construct TXS.GFP-SERI-CP accumulated similar levels of CP to constructs TXS.GFP-IRES-CP and TXS.GFP-IRESs-CP. In addition, TXS.GFP-HIRES-CP, which showed slightly reduced cell to cell movement on plants relative to the other IRES containing constructs, showed the lowest level of CP accumulation in protoplasts out of the constructs that produced any CP.

TABLE 2
Green fluorescent protein and coat protein
accumulation in N. benthamiana protoplasts.**

| Inoculum | ng GFP/μg TSP | ng CP/μg TSP |
|---|---|---|
| TXS | 0.0 | 0.492 |
| TXS.GFP | 0.0369 | 0.442 |
| TXS.GFP-ΔCP | 0.0603 | 0.0 |
| TXS.GFP-IRES-CP | 0.0360 | 0.1518 |
| TXS.GFP-IRESs-CP | 0.0342 | 0.1080 |
| TXS.GFP-SERI-CP | 0.0415 | 0.1081 |
| TXS.GFP-HIRES-CP | 0.0347 | 0.0504 |
| TXS.GFP-IRESH-CP | 0.0428 | 0.0 |
| MOCK | 0.0 | 0.0 |

**Protoplasts were inoculated with transcripts and levels of GEP and CP accumulation were assayed by ELISA, 2 dpi. TSP = total soluble protein.

### EXAMPLE 2

Method for Improving/Screening IRES Activity.

To mutate the IREScp sequence in pTXS.GFP-IRESs-CP the plasmid was used in mutagenic PCR basically as described by Leung et al. [25] with a 5′ primer equivalent to the 3′ end of the GFP gene (GES) and a 3′ primer complementary to the 5′ end of the PVX coat protein gene (N3#4). The products of a first round of mutagenic PCR were used as template in a second round of mutagenic PCR. The amplification products from the second round PCR were digested with SalI and SacI and ligated into the progenitor plasmid, digested with the same enzymes, in place of the unmutated IREScp sequence. After transformation into E. coli, DNA was prepared from a population of circa 100,000 independent clones and used as template in a further two rounds of mutagenic PCR. The final amplification products were digested with SalI and SacI and ligated into like cut pTXS.GFP-IRESs-CP. After transformation into E. coli, DNA was prepared from a population of circa 200,000 independent clones. Further, DNA was prepared from six individual clones for nucleotide sequence determination of the mutated sequence, which indicated a base mutation rate of approximately 7%.

To select clones with modified IRES activity the DNA population was linearized with SpeI and transcripts inoculated to N. benthamiana. Plants were inspected at 7 dpi under UV illumination and two variant lesions displaying increased fluorescence/larger fluorescent lesion area selected. The lesions were excised and RNA extracted [1]. The RNA was reverse transcribed according to the manufacturers instructions with SuperScript II (GIBCO BRL, Paisley, U.K.) and a primer complementary to the 5′ end of the PVX CP gene (N3#4). The first strand cDNA products were purified and amplified through PCR using a primer equivalent to the 3′ end of the GFP gene and a primer complementary to the 5′ end of the PVX CP gene. The amplification products, encompassing the mutated IREScp sequence, were digested with SalI and SacI prior to cloning between the same sites of pTXS.GFP-IRESs-CP producing SC196 and SC197.

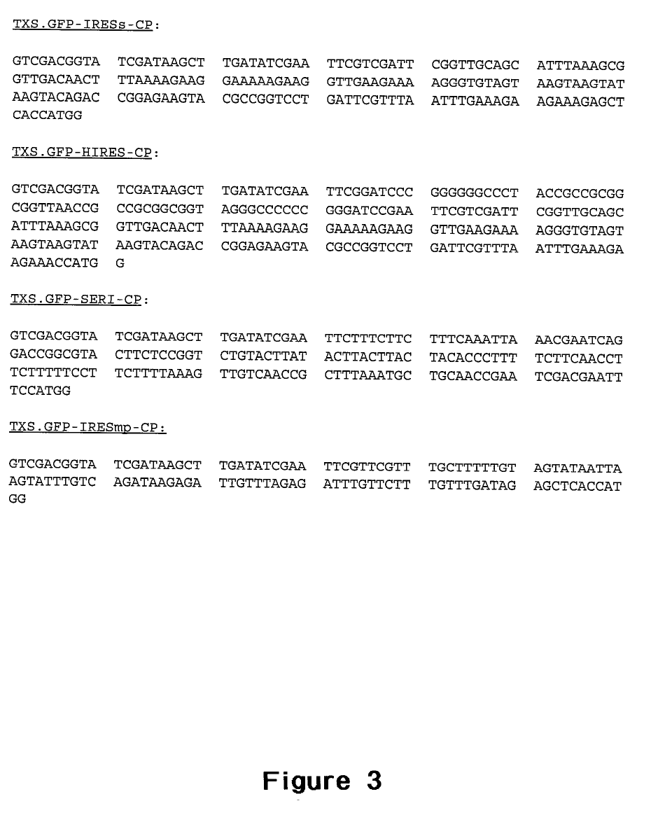

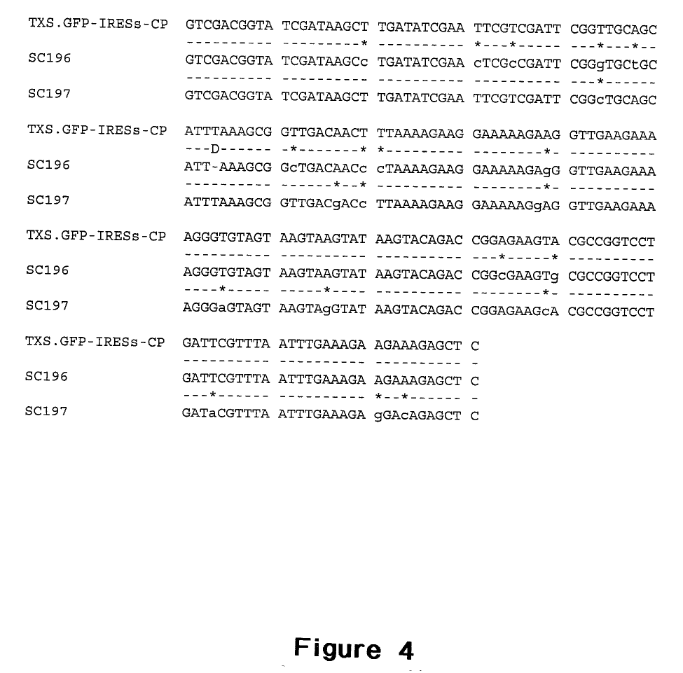

To test whether clones with enhanced rates of cell to cell movement had been obtained, resulting from increased levels of IREScp activity, DNA was linearized and transcribed. The transcripts were inoculated to one half of N. benthamiana leaves. Transcripts from the unmodified progenitor clone were inoculated to the other halves of the leaves as a control. Lesion area measurements were performed as described in methods. The mean lesion areas were calculated for the sample and control inoculations on paired half leaves (Table 3). Two-sample T-tests showed that both of the selected clones produced significantly larger lesions than the unmodified control (p>0.1). The nucleotide sequence of the mutated IREScp sequence found in the clones SC196 and SC197 was determined (FIG. 4). Alignment of the determined sequences with the unmodified sequence of the progenitor clone, pTXS.GFP-IRESs-CP, between the SalI and SacI sites used for cloning of the mutated fragment showed base mutation rates of 6.7 and 5.9% respectively (FIG. 3).

TABLE 3
Measurement of fluorescent infection foci.***

| | Mean area of fluorescent |
|---|---|
| Inoculum | infection foci (mm²) |
| SC196 | 1.10 |
| TXS.GFP-IRESs-CP | 0.492 |
| SC197 | 1.74 |
| TXS.GFP-IRESs-CP | 0.547 |

***The areas of fluorescent lesions on inoculated half leaves of N. benthamiana were measured at 8 dpi. Mean areas in mm² are presented for paired half leaves inoculated with samples and control.

Most approaches to foreign gene expression using virus-based vectors have relied either on the synthesis of a polyprotein, which is proteolytically processed to release the foreign protein, or depend on a viral promoter to direct expression of a foreign gene at the 5′-end of a subgenomic mRNA. Here we have investigated the use of internal initiation of translation as an alternative approach to the expression of genes from virus vectors. The use of an IRES sequence to direct gene expression avoids the need to duplicate promoter sequences in gene insertion, circumventing the possibility of homologous recombination. We used PVX-based vector constructs that produce a bicistronic mRNA containing the IREScp sequence previously described by Ivanov et al. [15] in which expression of the 3′-proximal CP gene was dependent on internal ribosome entry (FIG. 2, Tables 1 and 2). All constructs carrying the gfp gene accumulated similar levels of GFP with the exception of TXS.GFΔCP, which accumulated two- to three-fold more GFP than the other vectors. This observation is most likely a consequence of the higher transcriptional activity of the single, 3′-proximal, subgenomic promoter present in this vector.

A stable stem loop structure inserted at the 5′ end of the IREScp sequence did not abolish expression of the CP indicating that leaky scanning of ribosomes through the gfp gene was unlikely. In addition a construct in which the IREScp sequence was completely deleted gave rise to single cell infection sites further suggesting that leaky scanning was not occurring. In contrast to the situation with the stem loop positioned 5′ to the IREScp the stem loop structure inserted 3′ to the IREScp sequence prevented expression of the downstream CP open reading frame in the bicistronic mRNA. Thus the stem loop was able to effectively block scanning ribosomes. These results indicate that the IREScp sequence is functioning as a site for direct recruitment of ribosomes for initiation of translation. Our results support observations by Skulachev et al [16] that 3′-proximal gene expression was obtained from bicistronic transcripts, separated by the IREScp sequence, even when translation of the first gene was abolished by a stem loop structure inserted upstream of the 5′-proximal open reading frame. However, in our experiments the presence of the stem loop immediately upstream of the IRES sequence led to a reduction in CP expression levels (FIG. 2, Tables 1 and 2). This observation could be explained if the upstream loop sequence interfered with direct ribosome landing at the IRES, a conclusion consistent with the known importance of tertiary structure in some animal viral and cellular IRES sequences [11], resulting in reduced levels of CP expression.

Alternatively, reinitiation of translation, a phenomenon previously reported to occur with low efficiency in plants [23], could explain translation of the downstream ORF in constructs carrying the putative IRES sequence. In this scenario actively translating ribosomes might be capable of inefficiently melting the loop structure prior to reaching the GFP stop codon. That reinitiation of translation is resulting in expression of the CP is argued against by the fact that the levels of accumulation of CP, encoded by the 3′ cistron of the bicistronic mRNA, are higher than those of GFP, though, this does not take into account possible differences in protein stability between the GFP and CP. Also, it has been shown that IRES sequences themselves can be strong inhibitors of translation by the scanning mechanism, due to their highly structured nature [24]. Surprisingly, the IREScp sequence appears to initiate translation effectively in either orientation indicating that either the IRES activity is not orientation specific or that there may be a functional structure that is conserved in both strands. An alternative explanation is that there is a cryptic promoter element within the IREScp sequence that is able to initiate transcription of an additional subgenomic RNA, however, we obtained no evidence of extra subgenomic RNAs in northern blotting experiments and there is no reason to expect a tobamovirus-derived sequence to function as a promoter when present in a heterologous virus.

This ability of the IREScp sequence to function in either orientation raises the possibility that this sequence does not function in the same fashion as previously described IRES sequences from animal viruses [11]. Further experiments are required to define precisely the mechanism(s) by which the IREScp sequence acts. However, it is possible that both internal initiation and re-initiation of translation are operating concurrently, and when one system is abolished the other is able to continue to drive translation of the downstream gene.

The data presented demonstrates using a plant virus-derived IRES to direct translation of a protein from a heterologous viral vector.

Although the invention has been described with reference to the presently preferred embodiments, it should be understood that various modifications can be made without departing from the spirit of the invention.

All publications, patents, patent applications, and web sites are herein incorporated by reference in their entirety to the same extent as if each individual patent, patent application, or web site was specifically and individually indicated to be incorporated by reference in its entirety.

### LITERATURE CITED

1. Chapman, S. N., Kavanagh, T. and Baulcombe, D. C (1992) Plant J. 2, 549-557.

2. Dolja, V. V., McBride, H. J. and Carrington, J. C. (1992) Proc. Natl. Acad. Sci. USA 89, 10208-10212.

3. Scholthof, H. B., Morris, T. J. and Jackson, A. O. (1993) Mol. Plant-Microbe Interact. 6, 309-322.

4. Dolja, V. V., Haldeman, R., Robertson, N. L., Dougherty, W. G. and Carrington, J. C. (1994) EMBO J. 13, 1482-1491.

5. Baulcombe, D. C., Chapman, S. N. and Santa Cruz, S. (1995) Plant J. 7, 1045-1053.

6. Turpen, T. H. (1999) Phil. Trans. Royal Soc. B Series 354, 665-673.

7. Takamatsu, N., Watanabe, Y., Yanagi, H., Tadayoshi, S. and Okada, Y. (1990) FEBS Lett. 269, 73-76.

8. Hamamoto, H., Sugiyama, Y., Nakagawa, N., Hashida, E., Matsunaga, Y., Takemoto, S., Watanabe, Y. and Okada, Y. (1993) Bio-Technol. 11, 930-932.

9. Donson, J., Kearney, C. M., Hilf, M. E. and Dawson, W. O. (1991) Proc. Natl. Acad. Sci. USA 88, 7204-7208.

10. Kumagai, M. H., Turpen, T. H., Weinzettl, N., della-Cioppa, G., Turpen, A. M., Donson, J., Hilf, M. E., Grantham, G. L., Dawson, W. O., Chow, T. P., Piatak, M and Brill, L. K. (1993) Proc. Natl. Acad. Sci. USA 90, 427-430.

11. Dawson, W. O., Lewandowski, D. J., Hilf, M. E., Bubrick, P., Raffo, A. J., Shaw, J.J., Grantham, G. L. and Desjardins, P. R. (1989) Virol. 172, 285-292.

12. Kozak, M. (1991) J. Biol. Chem. 266, 19867-19870.

13. Houdebine, L. M. and Attal, J. (1999) Transgenic Res. 8, 157-177.

14. Belsham, G. J. and Lomonosoff, G. P. (1991) J. Gen. Virol. 72, 3109-3113.

15. Ivanov, P. A., Karpova, O. V., Skulachev, M. V., Tomashevskaya, O. L., Rodionova, N. P., Dorokhov, Y. L. and Atabekov, J. G. (1997) Virol. 232, 32-43.

16. Skulachev, M. V., Ivanov, P. A., Karpova, O. V., Korpela, T., Rodionova, N. P., Dorokhov, Y. L. and Atabekov, J. G. (1999) Virol. 263, 139-154.

17. Koenig, R. and Leseman, D. -E. (1989) in: AAB Descriptions of Plant Viruses (Murrant, A. F. and Harrison, B. D., Eds.) No. 354, pp. 1-5, Association of Applied Biologists, Warwick.

18. Santa Cruz, S., Chapman, S, Roberts, A. G., Roberts, I. R. Prior, D. A. M., Chapman, S. and Oparka, K. J. (1995) Proc. Natl. Acad. Sci. USA 93, 6286-6290.

19. Santa Cruz, S., Roberts, A. G., Prior, D. A. M., Chapman, S. and Oparka, K. J. (1998) Plant Cell 10, 495-510.

20. Power, J. B. and Chapman, J. V. (1985) in: Plant Cell Culture (Dixon, R. A. ed.), pp. 37-66, IRC Press, Oxford.

21. Gal-On, A., Kaplan, I. B., Roossink, M. J. and Paulukaitis, P. F. (1994) Virol. 205, 280-289.

22. Clarke, M. F. and Bar-Joseph, M. (1984) in: Methods in Virol. (Maramorsoch, K. and Koprowski, H. eds.), vol. 7, pp. 51-85, Academic Press, San Diego.

23. Futterer, J and Hohn, T (1996) Plant Mol. Biol. 32, 159-189.

24. Havenga, M. J. E., Vogels, R., Braakman, E., Kroos, N., Valerio, D., Hagenbeek, A. and van Es, H. H. G. (1998) Gene 222, 319-327.

25. Leung, D. W., Chen, E. and Goeddel, D. V. (1989). Technique 1, 11-15.

What is claimed is:

1. A polynucleotide comprising: (1) an IRES nucleotide sequence, (2) an ORF encoding a peptide of interest, and (3) an ORF encoding a viral protein, where (1) is located between (2) and (3).

2. The polynucleotide according to claim 1 wherein a promoter 5′ to (1), (2) and (3) transcribes a mRNA containing (1), (2) and (3).

3. The polynucleotide according to claim 2 wherein the IRES nucleotide sequence is a naturally occurring IRES or a fragment of a naturally occurring IRES that can direct translation of (2) or (3).

4. The polynucleotide according to claim 2 wherein the IRES sequence comprises a nucleotide sequence of: SEQ ID NO: 1; SEQ ID NO: 2; SEQ ID NO: 3; SEQ ID NO: 4; SEQ ID NO: 5; SEQ ID NO: 6; SEQ ID NO: 7; or a fragment of SEQ ID NO: 1, SEQ ID NO: 2, SEQ ID NO: 3, SEQ ID NO: 4, SEQ ID NO: 5, or SEQ ID NO: 6, or SEQ ID NO: 7, that can direct translation of (2) or (3).

5. The polynucleotide according to claim 2 wherein the viral protein is a coat protein.

6. A recombinant viral vector comprising a polynucleotide according to claim 1.

7. A recombinant virus comprising a recombinant viral vector according to claim 5.

8. A host comprising a recombinant virus according to claim 6.

9. An IRES capable of directing the expression of an internal ORF in a heterologous viral vector.

10. An IRES according to claim 9 wherein the IRES is a IREScp.

11. An IRES according to claim 10 wherein the IRES is crTMV IREScp.

12. A viral vector construct that expresses a bicistronic mRNA comprising an ORF positioned upstream of an IRES sequence and followed by a coat protein coding sequence.

13. A viral vector construct according to claim 12 wherein the ORF encodes a native or foreign gene.

14. A viral vector construct according to claim 13 wherein the reporter gene encodes a green fluorescent protein.

15. A viral vector construct, comprising: (1) a viral genome, and (2) an IRES sequence, wherein the IRES sequence is heterologous to the viral genome, wherein the IRES sequence is downstream of a desired gene or ORF and upstream of a virus coat protein gene, wherein the IRES sequence is in the sense or antisense orientation.

16. A viral vector construct according to claim 15 wherein the viral vector construct expresses a bicistronic mRNA.

17. A viral vector construct according to claim 15 wherein the viral genome is the genome of potato virus X.

18. A potato virus X-based viral vector construct comprising the viral vector construct according to claim 15, wherein the potato virus X-based viral vector construct gives rise to single cell infection sites.

19. A viral vector construct according to claim 15 further comprising (3) a stable stem loop structure inserted 5′ of the IRES sequence.

20. A viral vector construct according to claim 19 wherein the stem loop structure is immediately upstream of the IRES sequence.

21. A viral vector construct according to claim 20 wherein the stem loop structure causes a reduction in the expression of the virus coat protein gene.

22. A viral vector construct according to claim 21 wherein the stem loop structure interferes with direct interaction of a ribosome at the IRES sequence.

23. A viral vector construct according to claim 15 further comprising (3) a stable stem loop structure inserted 3′ of the IRES sequence.

24. A viral vector construct according to claim 23 wherein the stem loop structure prevents expression of the virus coat protein gene.

25. A viral vector construct according to claim 23 wherein the stem loop structure effectively blocks scanning ribosomes.

26. A viral vector comprising a plant virus-derived IRES sequence linked to the ORF encoding a protein of interest, wherein the plant virus-derived IRES sequence directs translation of the ORF and wherein the protein of interest is heterologous to the viral vector.

27. A viral vector according to claim 26 wherein the plant virus-derived IRES sequence initiates translation effectively in either sense or antisense orientation.

28. A viral vector according to claim 27 wherein the plant virus-derived IRES sequence is an IREScp sequence.

29. A viral vector construct comprising the function of producing a bicistronic subgenomic RNA in which two ORFs are separated by an IRES.

30. A viral vector construct comprising a modified IRES sequence that directs higher levels of protein expression.

31. A nucleic acid construct comprising a bicistronic message with an intervening IRES sequence.

32. A transgenic virus comprising the nucleic acid construct according to claim 31.

33. A transgenic virus comprising a foreign IRES.

34. A method of regulating the rate at which a virus infection spreads in a host, comprising: placing a nucleic acid construct comprising an internal ribosomes entry site upstream of a coat protein gene, wherein the internal ribosome entry site is chosen by the rate of infection of the viral vector on a host in the presence of that IRES.

35. A method of directing the expression of a foreign nucleic acid sequence in a host in the absence of multiple subgenomic promoters in a virus, comprising: placing a nucleic acid construct comprising an internal ribosomes entry site upstream of a foreign gene.

36. A method of directing the expression of a foreign ORF in a host, comprising:
(a) inserting a nucleic acid construct comprising a bicistronic message with an intervening IRES into a virus;
(b) infecting a host with the virus; and
(c) growing the host;
whereby the foreign ORF is expressed.

37. A potato virus X-based viral vector construct having the designation TXS.GFPΔCP.

38. A polynucleotide comprising pIRESs-XCP.

39. A polynucleotide comprising pIRES-XCP.

40. A polynucleotide comprising pSERI-XCP.

41. A polynucleotide comprising pHIRES-XCP.

42. A polynucleotide comprising pTXS.GFP.IRES-CP.

43. A polynucleotide comprising pTXS.GFP.IRESs-CP.

44. A polynucleotide comprising pTXS.GFP.SERI-CP.

45. A polynucleotide comprising pTXS.GFP.HIRES-CP.

46. A polynucleotide comprising pTXS.GFP.IRESH-CP.

47. A polynucleotide comprising pTXS.GFP-IRESs(mp)-CP.

48. A viral vector construct comprising TXS.GPF-IRES-CP.

49. A viral vector construct comprising TXS.GPF-IRESs-CP.

50. A viral vector construct comprising TXS.GPF-HIRES-CP.

51. A viral vector construct comprising TXS.GPF-IRESH-CP.

52. A viral vector construct comprising TXS.GPF-SERI-CP.


SEQUENCE LISTING (8 sequences)

SEQ ID NO: 1 - DNA, 188 bp, Potato virus
gtcgacggtatcgataagcttgatatcgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaagagctcaccatgg

SEQ ID NO: 2 - DNA, 251 bp, Potato virus
gtcgacggtatcgataagcttgatatcgaattcggatcccggggggccctaccgccgcggcggttaaccgccgcggcggtagggccccccgggatccgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaccatgg

SEQ ID NO: 3 - DNA, 187 bp, Potato virus
gtcgacggtatcgataagcttgatatcgaattctttcttctttcaaattaaacgaatcaggaccggcgtacttctccggtctgtacttatacttacttactacacccttttcttcaaccttctttttccttcttttaaagttgtcaaccgctttaaatgctgcaaccgaatcgacgaatttccatgg

SEQ ID NO: 4 - DNA, 122 bp, Potato virus
gtcgacggtatcgataagcttgatatcgaattcgttcgtttgctttttgtagtataattaagtatttgtcagataagagattgtttagagatttgttctttgtttgatagagctcaccatgg

SEQ ID NO: 5 - DNA, 181 bp, Potato virus
gtcgacggtatcgataagcttgatatcgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaagagctc

SEQ ID NO: 6 - DNA, 180 bp, Potato virus
gtcgacggtatcgataagcctgatatcgaactcgccgattcgggtgctgcattaaagcggctgacaaccctaaaagaaggaaaaagagggttgaagaaaagggtgtagtaagtaagtataagtacagaccggcgaagtgcgccggtcctgattcgtttaatttgaaagaagaaagagctc

SEQ ID NO: 7 - DNA, 181 bp, Potato virus
gtcgacggtatcgataagcttgatatcgaattcgtcgattcggctgcagcatttaaagcggttgacgaccttaaaagaaggaaaaaggaggttgaagaaaagggagtagtaagtaggtataagtacagaccggagaagcacgccggtcctgatacgtttaatttgaaagaggacagagctc

SEQ ID NO: 8 - DNA, 70 bp, Potato virus
aattcggatcccggggggccctaccgccgcggcggttaaccgccgcggcggtagggccccccgggatccg